(19) United States
(12) Patent Application Publication
(10) Pub. No.: US 2013/0186406 A1
(43) Pub. Date: Jul. 25, 2013

(54) GAS DELIVERY MASK FOR MEDICAL USE

(71) Applicants: Julius Hajgato, Shanty Bay (CA); Lee McDonald, Barrie (CA)

(72) Inventors: Julius Hajgato, Shanty Bay (CA); Lee McDonald, Barrie (CA)

(73) Assignee: SOUTHMEDIC INCORPORATED, Barrie, ON (CA)

(21) Appl. No.: 13/823,692

(22) PCT Filed: Nov. 30, 2011
(86) PCT No.: PCT/CA11/50741

§ 371 Date: Mar. 14, 2013

(51) Int. Cl. A61M 16/06

(52) U.S. Cl. CPC A61M 16/06
USPC 128/206.28

(57) ABSTRACT

There is provided a mask for delivery of a medical gas to a patient comprising a mask body having a rim for contacting the patient's face and an interior space configured to surround and enclose the patient's nose and mouth. The mask includes a gas diffuser structure projecting through said mask body into said interior space. The gas diffuser structure includes a support, a gas delivery tube within said support having an internal bore the axis of which is generally vertical when said mask body is upright, an inlet in said gas tube for communication with a source of pressurized gas, and an outlet from said gas tube within said interior space. The structure includes a gas flow disrupter or baffle located over the outlet of said bore in the path of gas exiting said bore configured to diffuse and/or redirect said gas radially outwardly. A flow of gas entering the mask forms a plume of enriched gas at the patient's nose and mouth region, thereby permitting a relatively high oxygen content to be delivered to the patient at a lower flow rate.

### RELATED APPLICATIONS

This application claims the benefit of U.S. Provisional Application No. 61/418,489 filed on Dec. 1, 2010 and U.S. Provisional Application No. 61/437,116 filed on Jan. 28, 2011, both of which are herein incorporated by reference.

### FIELD OF THE INVENTION

The present invention relates to medical devices, in particular a mask for delivering oxygen or other gas to a patient, in which the mask includes an improved gas inlet structure for discharging and dispersing gas within the mask interior from a source of pressurized gas.

### BACKGROUND OF THE INVENTION

Oxygen masks are employed for numerous medical uses to deliver a concentrated gas, typically oxygen or oxygen-enriched air, to patients. The mask is typically used by a patient for an extended period, for example to provide supplemental oxygen on long-term basis to a compromised patient. The needs of such a patient require the delivery of a relatively high concentration of oxygen (or other gas) in a stable, efficient fashion that minimizes patient discomfort. In order to improve efficiency, the flow rate should be as low as possible while still maintaining the required gas concentration at the patient's nose and mouth. As well, an overly high flow rate can result in patient discomfort, as the discharged gas impacts against the patient's face and leak from the mask to contact the patient's eyes.

For some applications, the mask should enable the health care professional to monitor the content of CO₂ and other gasses exhaled by the patient. For this purpose, the mask should provide an accurate sample of the patient's exhaled breath.

In a patient mask, oxygen concentration measured at the patient's nose is expressed as FiO₂%. Certain known masks, such as the Capnoxygen™ mask manufactured by Southmedic Inc., have been measured to deliver oxygen at a maximum concentration of 55-60 FiO₂%, with this level being achieved by employing relatively high flow rates of 8 LPM or more.

A medical mask can optionally be provided with a C0₂ monitoring tube for withdrawing a sample of exhaled breath from the patient. This permits monitoring of the C0₂ or other gas content of the exhaled breath. The exhaled gas is discharged into a monitor which charts the CO₂ content (or other selected gas) in the patient's breath. Accurate monitoring of the patient's breath requires that the mask is configured to effectively separate the patient's breath from the flow of incoming oxygen. Since both of these gasses exist within the same space, it can be difficult to provide such a separation within the confines of a mask, such that a relatively high concentration of exhaled breath is sampled without minimal mixing with the incoming oxygen gas.

A variety of mask configurations and structures are known, including the Capnoxygen™ oxygen mask 1, which is illustrated in FIG. 1. The bodies of such masks are either substantially open (with large openings in relation to the mask surface area), fully enclosed, or of intermediate openness. The Capnoxygen mask is provided with a substantially enclosed mask body and an oxygen inlet opening into the interior of the mask body. The inlet constitutes a tube which enters a lower portion of the body, and is oriented vertically when the mask is upright, as seen in FIG. 1. The vertical orientation of the tube generates a flow of gas obliquely to the patient's face, such that the gas does not blow directly towards the patient. Rather, the gas flow is directed into the upper reaches of the mask interior, from where it is redirected downwardly towards the patient's nose and mouth. The Capnoxygen mask also includes a C0₂ monitoring tube which opens into the mask body. The monitoring tube is a vertically-oriented tube located parallel to the inlet tube, and spaced apart from the inlet tube to ensure that the gas withdrawn into the tube contains at least a reasonably high fraction of exhaled air.

It is desirable to provide an improved mask that provides a relatively high Fi0₂%, even at relatively low oxygen flow rates. It is also desirable to provide a mask that includes a C0₂ monitoring component that is structured and configured to provide relatively broad peaks of exhaled C0₂.

### SUMMARY OF THE INVENTION

According to one aspect, the invention relates to a mask for delivery of a medical gas to a patient. The mask comprises a substantially enclosed mask body having a rim for contacting the patient's face and an interior space configured to surround and enclose the patient's nose and mouth. The mask further includes a gas diffuser structure projecting through said mask body into said interior space. The gas diffuser structure includes a gas discharge tube having an internal bore which is generally vertical when said mask body is upright. The discharge tube has an inlet for communication with a source of pressurized gas and an outlet located within the mask interior space. The mask further includes a baffle comprising a gas strike surface located over the outlet of said bore in the path of gas exiting said bore.

The baffle is configured to efficiently direct inflowing gas radially outwardly from the gas diffuser, so as to form a plume which is concentrated at the user's nose and mouth region. In one aspect, the baffle comprises a post mounted within said bore and a head supported by said post, an undersurface of said head comprising said gas strike surface. The baffle can be “mushroom shaped” whereby the gas strike surface is concave.

The baffle can be retained by a support projecting radially inwardly within said bore to seat said post within said bore. The support can comprise a sleeve or socket projecting upwardly from said bore to receive the post within said sleeve, said sleeve being inwardly spaced from the inside surface of said bore.

The baffle can be removeable from said bore, for example by removing the post from the sleeve, to thereby permit replacement of the baffle for one with a different configuration to provide a different gas distribution pattern.

The mask body can comprise a forwardly protruding snout portion, said snout portion having a generally horizontal floor wherein said gas diffuser structure projects through said floor into the interior of said snout portion.

The mask may include a conduit for sampling exhaled breath from a user. The conduit includes an inlet within the mask interior facing the user and an outlet for transmitting exhaled breath to an external gas monitor. The conduit can include a gas sampling tube projecting horizontally towards the user when the mask is upright, positioned below said outlet and having an inlet radially displaced therefrom towards the user's face. Alternatively, the inlet can comprise a port within said mask body, said port communicating with said conduit which can be positioned below said outlet and radially displaced therefrom towards the user's face.

The invention also relates to a kit of parts for a medical gas delivery mask, in which the kit comprises components as described above, provided in kit form for assembly into a mask as described herein.

The invention also relates to a method for delivering gas to a patient. The method includes the steps of providing a mask which defines a substantially enclosed space over the patient's nose and mouth region including a rim contacting the patient's face around the nose and mouth region, delivering a pressurized gas into the mask interior in through a tube which discharges gas in an upward direction when the mask is upright and deflecting the upwardly rising gas flow radially outwardly in a horizontal plane. Gas flow within the mask can be deflected by introducing the gas flow through a vertical conduit into the mask interior and positioning a baffle over the outlet of the conduit within the mask interior. The baffle has a gas strike surface facing the gas outlet to diffuse the gas into a plume extending radially outwardly from the diffuser, to form a gas-enriched plume at the user's nose and mouth region.

The present specification includes directional references such as “upright”, “horizontal” and the like. Such references are intended merely for convenience of description, and are not intended to limit the scope of the invention. It will be evident that the present mask may be oriented in any direction when used; for convenience, the mask is described throughout this application as orientated in an upright position, as if worn by a patient standing or sitting upright. In similar fashion, any dimensions or similar references are presented by way of example and are not intended to limit the scope of the invention, unless specifically stated to constitute an element of the invention or an embodiment thereof. In the present specification, reference to the use of the mask for specific gases is exemplary only. It is evident that the mask may be used for a supply of various gases to a patient, including oxygen but also other gases as required by the patient.

### BRIEF DESCRIPTION OF THE DRAWINGS

FIG. 1 is a side elevational view of a prior art oxygen mask.

FIG. 2 is a side sectional view of a mask according to the present invention.

FIG. 3 is a side elevation view of thereof.

FIG. 4 is a perspective view thereof, from the rear.

FIG. 5 is a side elevational view of the gas diffuser structure of the mask of FIG. 2.

FIG. 5A is a front elevational view of the diffuser structure.

FIG. 6 is a perspective view of the gas diffuser structure.

FIG. 7 is a top view of the gas diffuser structure, with the gas-deflecting baffle removed.

FIG. 8 is a sectional view of the gas diffuser structure.

FIG. 9 is a perspective view of a further embodiment of the invention.

FIG. 10 is a perspective view of the gas diffuser structure according to the embodiment of FIG. 9.

FIG. 11 is a sectional view of the gas diffuser structure of FIG. 9.

FIG. 12 is a schematic view of the mask of FIG. 9 showing gas flow patterns within the mask body.

FIGS. 13-19 depict experimental results relating to tests conducting with an embodiment of the invention and a control.

FIGS. 20-27 depict computer-modeling tests conducted on an embodiment the present invention and a control.

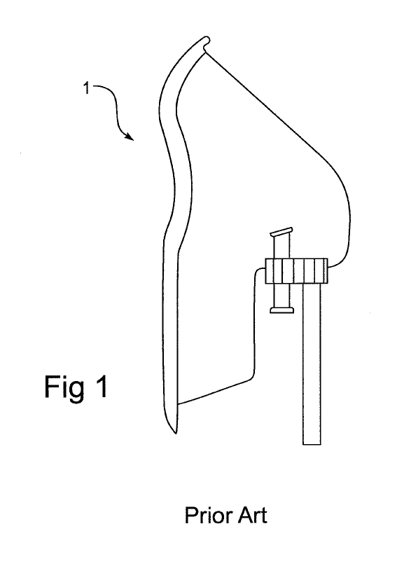

### DETAILED DESCRIPTION

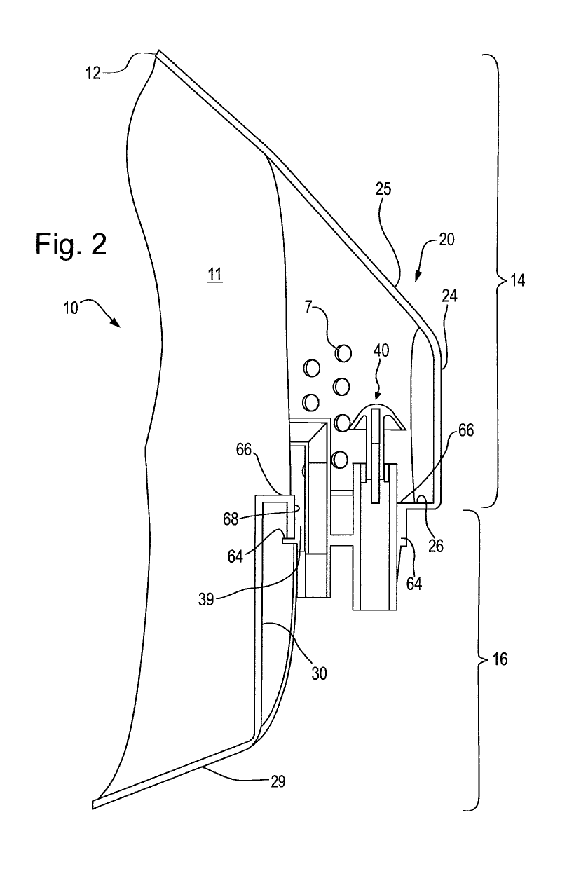

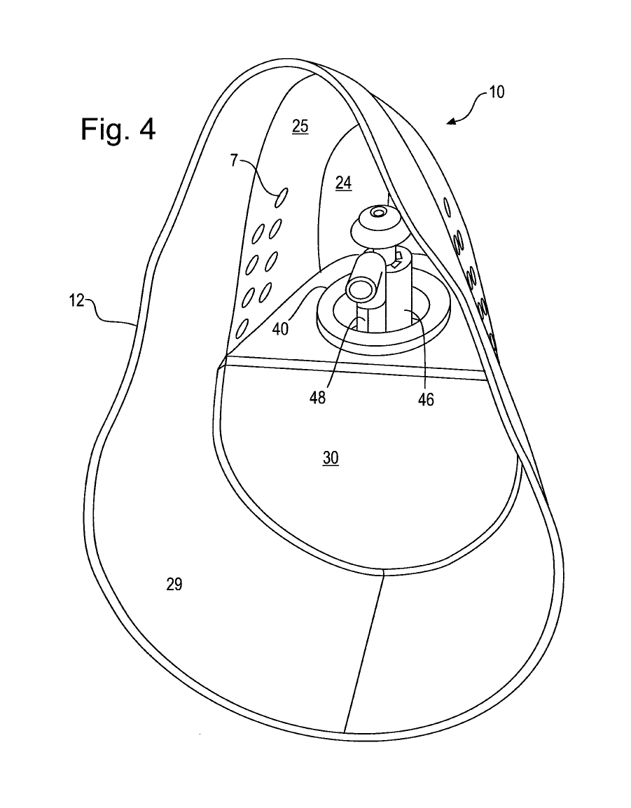

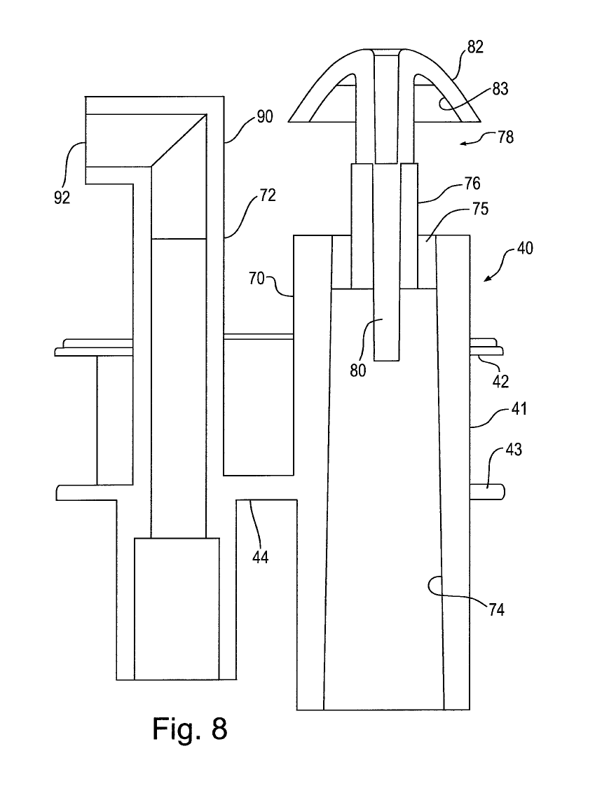

Referring to FIGS. 2-8, one embodiment of an oxygen mask 10 comprises a substantially closed mask body 11 configured to fit over the nose and mouth region of a patient. Mask body 11 defines an interior space configured to receive the user's nose, mouth and associated region of the user's face. Mask body 11 essentially encloses the patient's nose and mouth region, contacting the patient's face at rim 12 to provide a snug fit. Mask 10 includes perforations 7 located in the upper portion thereof for ventilation.

When oriented in a substantially vertical position (as illustrated and described herein), mask 10 comprises an upper region 14, which generally fits over the patient's nose and surrounding region and a lower region 16 configured to generally fit over the patient's mouth. Upper region 14 includes a forwardly-projecting snout 20. Snout 20 comprises a tapered sidewall 25, a substantially vertical front wall 24, and a flat floor 26 located at the base of snout 20. Lower mask region 16 is recessed from snout 20 and comprises a generally vertical front wall 30 the upper edge of which meets floor 26. A tapered inner sidewall 29 encircles mask and meets rim 12.

An opening extends through floor 26, defined by a tubular flange 60 which protrudes downwardly from the underside of floor 26. Flange 60 comprises lower and upper rims 64 and 66 respectively.

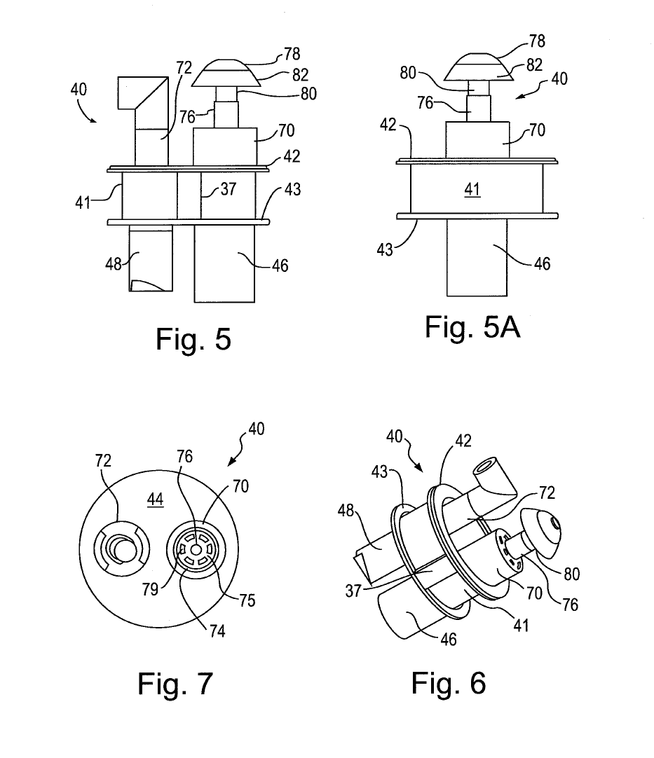

A gas diffuser structure 40, shown in detail in FIGS. 5-8 is retained at least partially within flange 60. Structure 40 comprises a cylindrical body 41 retained within the interior of flange 60. Body 41 includes a key 37 projecting from its outer surface, which fits within a slot 39 recessed into the interior surface of flange 60 to align body 41 within flange 60. Upper and lower laterally-projecting flanges 42 and 43 respectively project outwardly from the upper and lower edges of body 41. Flanges 42 and 43 overlap with upper and lower rims 66 and 64 respectively (as seen in FIG. 2), so as to tightly engage connector 40 within flange 60. Preferably, flanges 42 and 43 are flexible to permit connector 40 to assemble onto flange 60. Structure 40 may be retained purely by friction fit, or may be glued or otherwise fused to tube 60. Alternatively, structure 40 may be integrally moulded with mask body 11.

Gas diffuser structure 40 includes an interior shelf 44, contiguous with lower flange 43, which spans the interior of body 41. Gas tubes 46 and 48 pass through openings in shelf 44 and form an integral part of structure 40. Tubes 46 and 48 project downwardly from body 41 for connection to respective external gas conduits, not shown. Gas tube 46 is used for the supply of oxygen into mask 10, with the lower end thereof being configured for attachment to an oxygen supply conduit to discharge pressurized oxygen (or other gas) into the interior of mask 10. Tube 48 is configured for attachment to a CO₂ sampling conduit, for connection with a monitor to permit sampling of exhaled gases from the patient. Within the interior of mask 10, tubes 46 and 48 project upwardly from connector 40 to form interior stacks 70 and 72, for oxygen discharge and CO₂ sampling respectively.

A gas flow disrupter, consisting of a baffle 78, is fastened to stack 70 and projects upwardly from the upper end thereof. Baffle 78 includes an elongate post or stem 80 which is retained within a stem holder 76 located within bore 74 of stack 70. Stem holder 76 consists of a sleeve having an internal bore dimensioned to snugly retain stem 80. Stem holder 76 is spaced inwardly from bore 74 by supports 75, to provide an essentially annular space 79 for the discharge of gas around the outside of stem 80. The bore of stem holder 76 is tapered to match the taper of stem 80 to snugly retain baffle 78. Stem holder 76 projects upwardly from bore 74 to provide structural support for stem 80. Baffle 78 is generally similar in configuration to the gas flow disrupter incorporated within the SouthMedic Inc. OxyArm™ diffuser and as described in U.S. Pat. Nos. 6,450,166 and 6,631,719 (incorporated herein by reference). Baffle 78 is capped with a mushroom cap-shaped head 82. The lower surface of head 82, which is contacted by the gas stream emitted from bore 74, has a concave lower surface 83 facing bore 74. Stem 80 is wedged into holder 76 to extend into the interior of bore 74. Stem 80 may be retained in holder 76 solely by friction fit, or may be glued or otherwise fused into place. Annular gas discharge space 79 around stem 80 to permit the discharge of gas into the interior of mask 10 in an upward direction for contact with baffle 78. Head 82 projects laterally outwardly from stem 80, preferably at least co-extensively with the periphery of stack 70, or beyond. Gases discharged from bore 74 thus are discharged through annular opening 79. The discharged gases contact concave lower surface 83 of head 82, and are thus diverted outwardly in an essentially lateral fashion, in a plume or vortex that projects radially outwardly centred on a horizontal plane from baffle 78. The discharged gasses effectively form a “bubble” or plume of enriched gas projecting horizontally to contact the patient's nose and mouth region, with the area outside of the bubble comprising a region of lower oxygen concentration. As will be discussed below, this results in a relatively high effective FiO₂%, even at relatively low flow rates of oxygen entering the mask. The plume of discharged gas constitutes a region of turbulent gas flow, which is directed horizontally to contact the patient at the nose and mouth region, while impinging on the interior surface of the mask by a relatively small degree.

In one embodiment, bore 74 has an internal diameter of 0.24″ (0.61 cm), and stem 80 projects upwardly from stack 70 by 0.242″ (0.615 cm), as measured to the base of head 82.

Baffle 78 can be removed from holder 76, when provided with a friction fit engagement, to permit replacement of this component, for example to provide a different flow characteristic of the mask, when desired. However, in one alternative to the two piece structure described above, baffle 78 is formed with diffuser structure 40 as a single one-piece unit.

Turning to the CO₂ collector, stack 72 terminates in an elbow 90, having an open gas intake port 92 which projects rearwardly towards the patient's nose. Port 92 opens in a generally horizontal direction, in order to efficiently sample breath discharged from the patients nose or mouth. The CO₂ collector is configured to minimize interference between the plume of enriched incoming gas and the breath discharged by the patient. For this purpose, the CO₂ collector is configured to block the flow of oxygen to create a region of lower pressure and/or reduced oxygen concentration, where the user's breath will flow more easily into the collector without interference from the inflowing gas. This effect is illustrated graphically in the flow simulations in the present Figures.

The formation of the pressurized gas “bubble” or plume within the mask generates a vortex outside of the region of the bubble. The vortex comprises a region of relatively lower pressure to permit exhaled gas to enter port 92 without significant co-mingling with the incoming oxygen.

In operation, gas connections are engaged to tubes 46 and 48 for the supply of oxygen and sampling of exhaled breathe, respectively. The mask is fastened to the patient by any suitable means for covering the mouth and nose region, such that rim 12 snugly fits against the patient's face.

The mask is configured to provide a high FiO₂% percentage within a range of gas flow rates. In one example, the mask is able to provide an FiO₂% of approximately 70% at 8 LPM. In other embodiments, the mask is configured to provide a useful FiO₂% at gas flow rates of between 4 and 15 LPM, while redirecting at least a substantial portion of the incoming airflow towards the mouth and nose of the patient, rather than to the eyes. The configuration of the mask of the present invention provides a diffuser projecting upwardly, and with the curvature of the mask cooperating with the diffuser configuration and orientation to generate a concentrated plume of oxygen within the mask. The gas plume is substantially centred on the nose and mouth region of the patient within the mask interior and is at least partial out of contact with the mask body, particularly in the uppermost region where escaping gas may impact against the patient's eyes. The location and shape of the gas plume also permits the gas collector opening 92 to sample a breath sample that is relatively pure and unmixed with the gas discharged from gas tube 46.

The gas flow rate within mask 10 can be adjusted by altering the entrainment of ambient air in the gap between the diffuser and the face.

The oxygen concentration within the mask varies with the increase and decrease of flow rate and the axial distance from the diffuser to the face and the radial distance from the diffuser to a side wall. Concentration is approximately 1/concentration which varies with the distance from the diffuser, such that the concentration decreases as the distance of the diffuser from a surface and the rim of bore 74 increase. The dimensions described above have been found to provide an increased oxygen concentration and to minimize the disruption of the C0₂ flow to the outlet port.

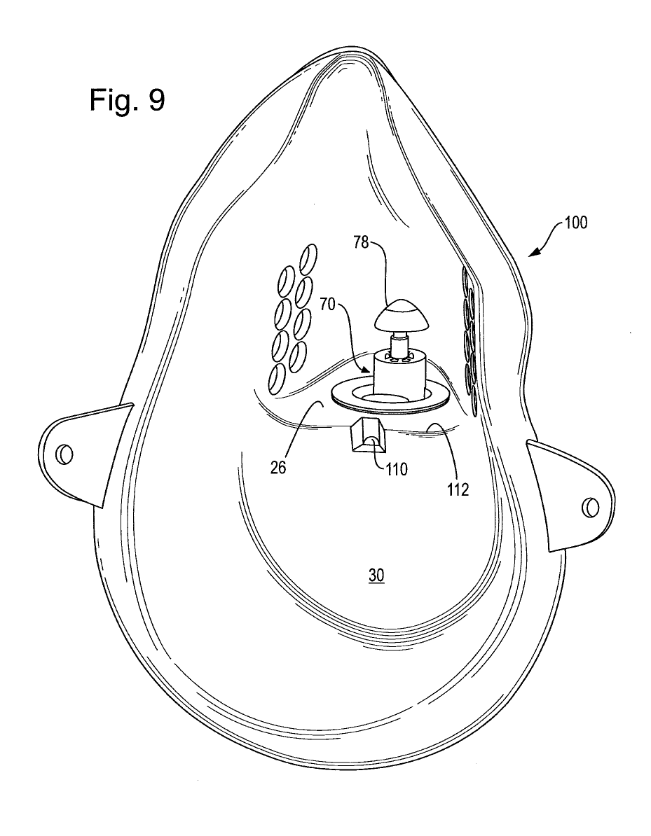

A further embodiment of the invention is illustrated in FIGS. 9 through 12. According to this embodiment, mask body 100 includes a slot-shaped breath-sampling port 110, recessed into the shoulder 112 where floor 26 and wall 30 of mask body meet. Port 110 opens into both floor 26 and wall 30, thereby opening to both upward and rearward directions, to admit gas flow from a range of directions. Port 110 flares outwardly towards its opening into the mask interior, and is intended to receive exhaled breath from the patient for sampling in a downstream gas analyzing system (not shown).

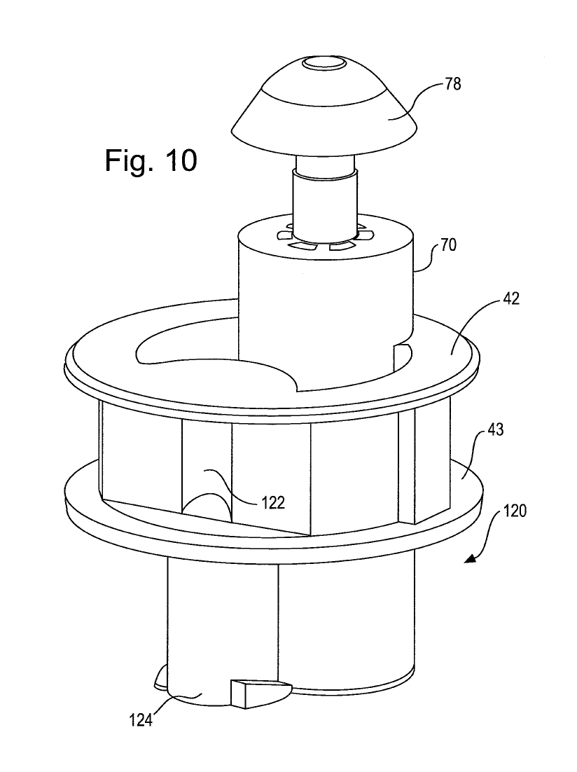

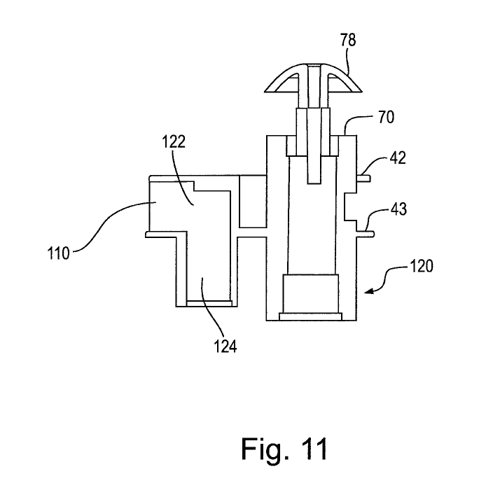

As seen in FIGS. 10 and 11, gas diffuser structure 120 in this embodiment comprises an interior stack 70, similar in configuration to the first embodiment, and gas disrupter baffle 78. Structure 120 comprises an opening 122, located between flanges 42 and 43. Opening 122 is located to align with port 110 within the mask body, such that exhaled gases may flow through port 110, directly into opening 122. Opening 122 communicates with gas tube 124 within structure 120, which is configured for attachment to a CO₂ sampling conduit in the same manner as tube 48 of the first embodiment.

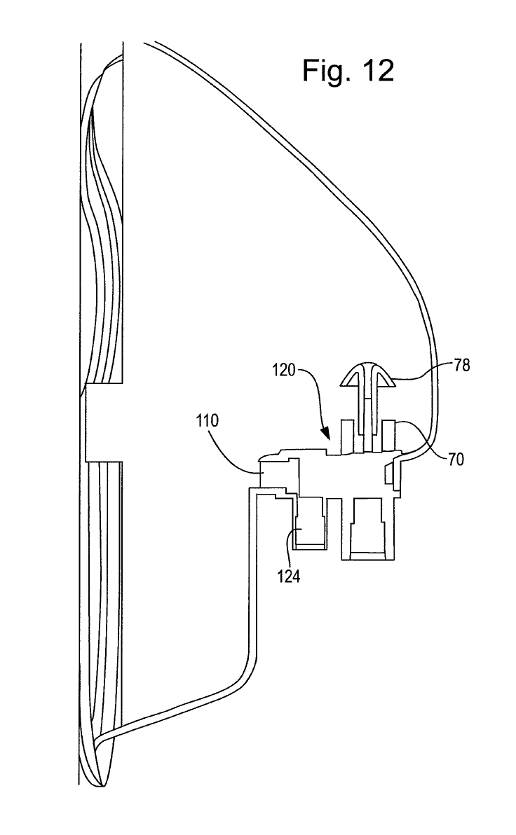

FIG. 12 graphically depicts gas flow patterns within the mask interior, in the mask of the embodiment of FIGS. 9-11. It will be seen that incoming gas is discharged generally horizontally from stack 70, in conjunction with baffle 78. Port 110 is located at some remove from the gas discharge region of stack 70, being located both below the level of gas discharge and radially (rearwardly) displaced from the location of gas discharge. Port 110 is thus located in a region of the mask body wherein there is normally a somewhat lower gas pressure when oxygen is discharged into the mask interior, relative to the plume of gas discharged from stack 70, thereby permitting exhaled breath to flow into port 110. The exhaled gas enters conduit 124 for sampling by a monitoring device (not shown).

### EXAMPLE 1

In a study, an embodiment of a mask according to the invention was tested on volunteer participants and compared with a prior art mask. Twenty three (23) Healthy adult subjects ranging in age from 19 to 61 years old were recruited to the study. The participants were seated upright. In order to simulate field conditions, no instruction was given to refrain from talking or to control breathing. Participants wore either a mask according to the present invention (referred to in the results below as the “new” mask) was used for the first series of tests or a prior art mask, consisting of a commercially available Capnoxygen mask (referred to as the “existing” mask).

It was determined that with a mask according to the invention, the flow rate in LPM available to the subject is close to the source flow rate, assuming velocities are equal, taking into account steady state equations only, defined as:

V2=V1*d12/d22=V3 (Concentration of Mass, Continuity)

X≈Q/2πKV 2Rrr2exp (−2r2/2Rr2) (Concentration of Mass, Gaussian Model)

K (constant) is what was resolved in relation to distance from the face or a surface or radial dispersion coefficient.

The mask was configured as a closed mask body to maximize oxygen concentration.

The sampling location was a sampling tube taped at the center lower lip of the study individual. A Datex-Ohmeda AS/5 multigas monitor had a sampling flow rate of 200 ml/min, and a delay time of approx. 2.5 s with this configuration. Alveolar gas equilibrium was achieved before stabilized waveforms were noted. Oxygen was supplied to the participants at 4, 6, 8, 10, 12 and 15 litres per minute (LPM). Flow rates were recorded as indicated by the Precession Medical oxygen regulator needle valve, model 31MFA10001 and through a Harris pressure regulator, model #9296. The tests were conducted as follows:
1) The new mask was attached to the participant's face and gas was discharged starting with a flow rate at 4 LPM. Five readings were taken and documented over 90 second intervals.
2) A sampling line was then connected to the mask sampling port, and attached to the Datex-Ohmeda monitor and a printout from the monitor that showed the participants C0₂ waveform at that time was taken and attached to the tabular records for analysis later.
3) The mask then was removed for 2 minutes from the participant's face, and the new setting was set on the flow meter at 6 LPM.
4) The mask was then re-attached to the participant's face.
5) Steps 1 through 4 were repeated for the flow rates 8, 10, 12 and 15 LPM.
6) The mask was removed and another mask (either the new or existing connector) was attached to the participants face.
7) Steps 1 through 5 were repeated. At the end of each session the participant was also asked about the comfort level. Their comments were also recorded.

Reported mean oxygen concentrations and associated standard deviations (SD) in the sample size for each flow rate are the results of at least 5 individual readings collected over 90 second intervals. Aggregate data was assembled from the results of the individual readings. Mean and standard deviation was calculated within each group.

The tests demonstrated that the tested embodiment provides a relatively high oxygen concentration and good C0₂ waveform, namely responsiveness to the user's exhalations for close to realtime measurement of the user's exhaled breath.

Low flow systems deliver 100% oxygen at flows that are less then the patient's inspiratory flow rate (i.e., the delivered oxygen is diluted with room air) and thus the oxygen concentration (FiO₂) may be high or low depending on a specific device and the patient's rate.

Nasal cannula can provide 24-40% oxygen with flow rates up to 6 L/min but should be humidified at rates above 4 L/min.

Gas deliver at rates higher then 6 to 10 LPM and 40-70% oxygen require a partial re-breathing mask. This is considered a low flow system; a non re-breathing mask is similar to the partial re-breathing mask, except that it has a series of one-way valves. This requires a minimum flow of 10 L/min. The delivered FiO₂ of this system is 60-80%.

The tests determined the following FiO₂ levels. The values expressed below represent the mean FiO₂ levels determined in the tests described above, for the existing and new masks, at flow levels between 4 and 15 LPM.

| Flow rate | | Existing Mask FiO₂ | New Mask FiO₂ |
|---|---|---|---|
| 4 | LPM | 34.9 | 50.9 |
| 6 | LPM | 41.7 | 62.3 |
| 8 | LPM | 44.1 | 68.9 |
| 10 | LPM | 49.2 | 74.2 |
| 12 | LPM | 49.6 | 77.2 |
| 15 | LPM | 55.4 | 81.8 |

It will be seen from the tests conducted herein that the FiO₂ values were consistently and significantly higher at gas flow levels between 4 and 15 LPM, for a mask according to the present invention as compared with a prior art example.

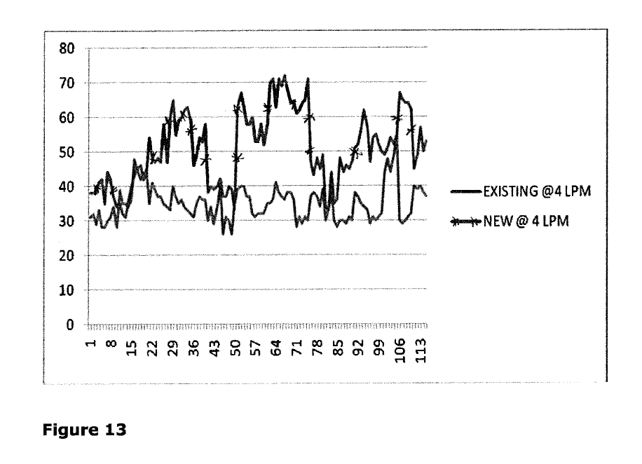

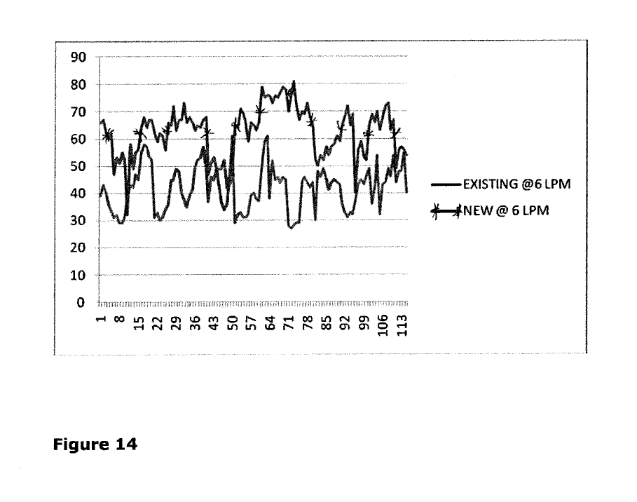

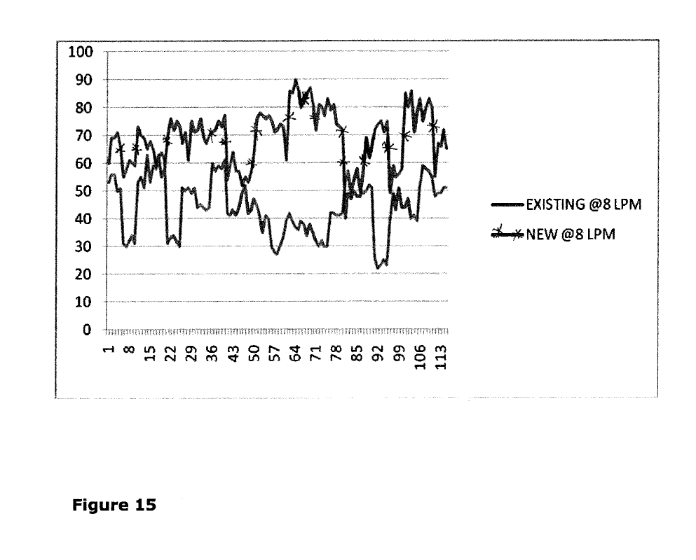

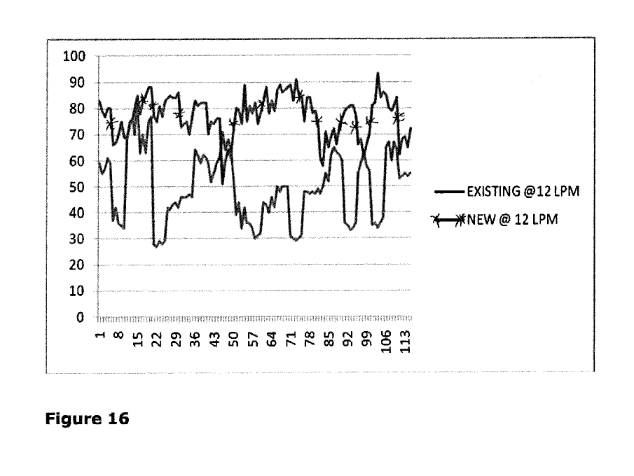

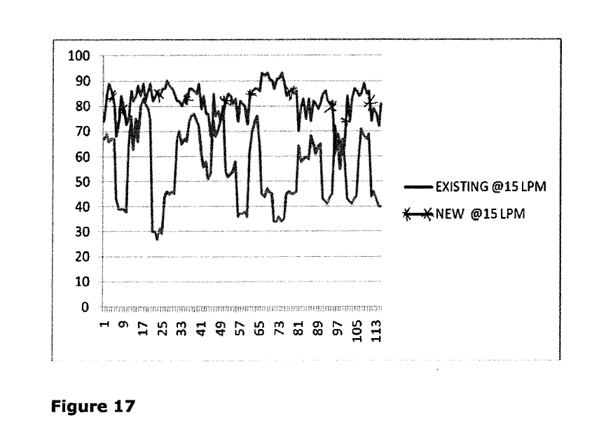

FIGS. 13-17 show the individual FiO₂ levels measured in respect of the tests described above.

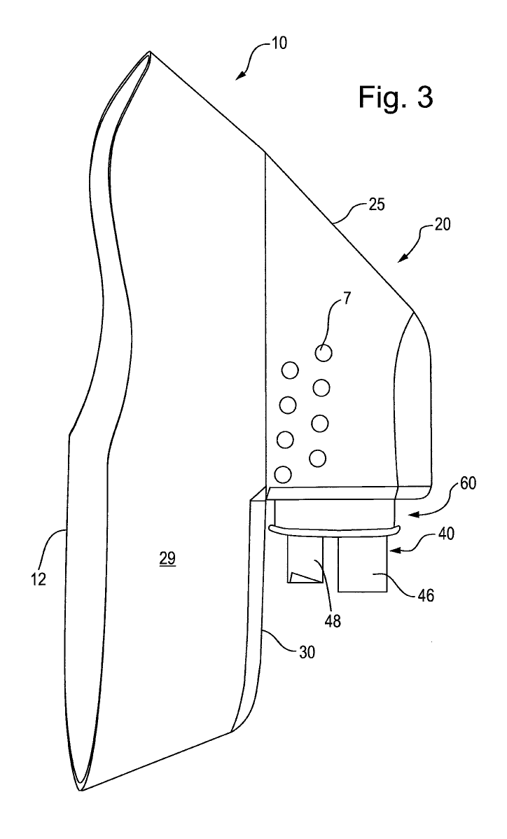

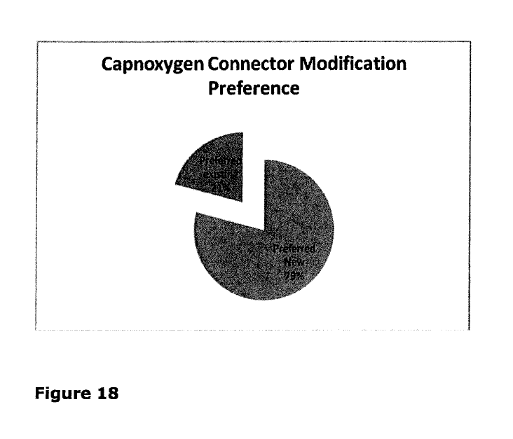

In general it is difficult for a single mask to provide a suitable level of performance for low flow situations as well as meet high flow requirements. The mask of the present invention is intended, in some embodiments, to be used in most situations without the need for the patient to switch to various devices during treatment. The present mask is intended in at least some embodiments to improves patient comfort as the design redirects the airflow towards the mouth and nose (refer to FIGS. 3, 4, 5 and 6) rather then to the eyes, as one of the main complaints received for the existing design is it bothers the eyes. FIG. 18 summarizes the results of the patient's comments in the present test.

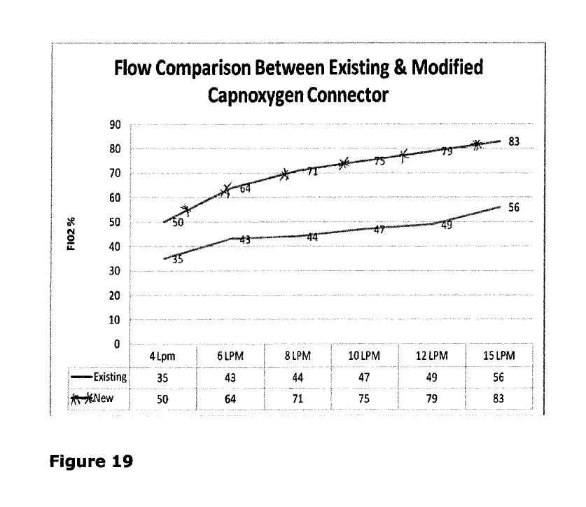

FIG. 19 summarizes the results above relating to FiO₂ levels determined in the above tests.

Since this mask would currently be targeted for flow of only 8 LPM, it be easily ascertained that improvement from 44 to 71% is quite dramatic (61% improvement).

### EXAMPLE 2

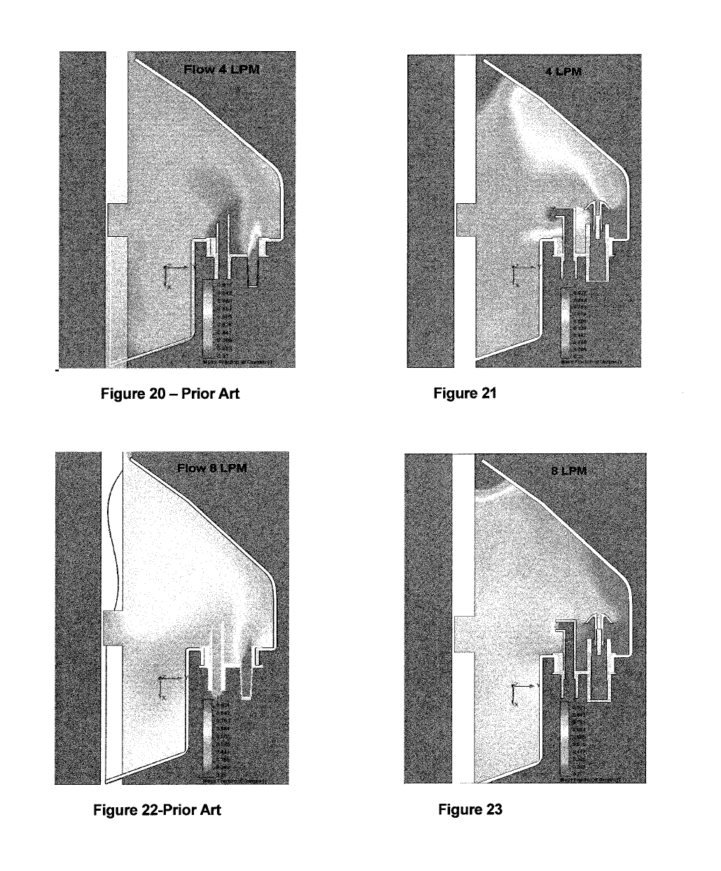

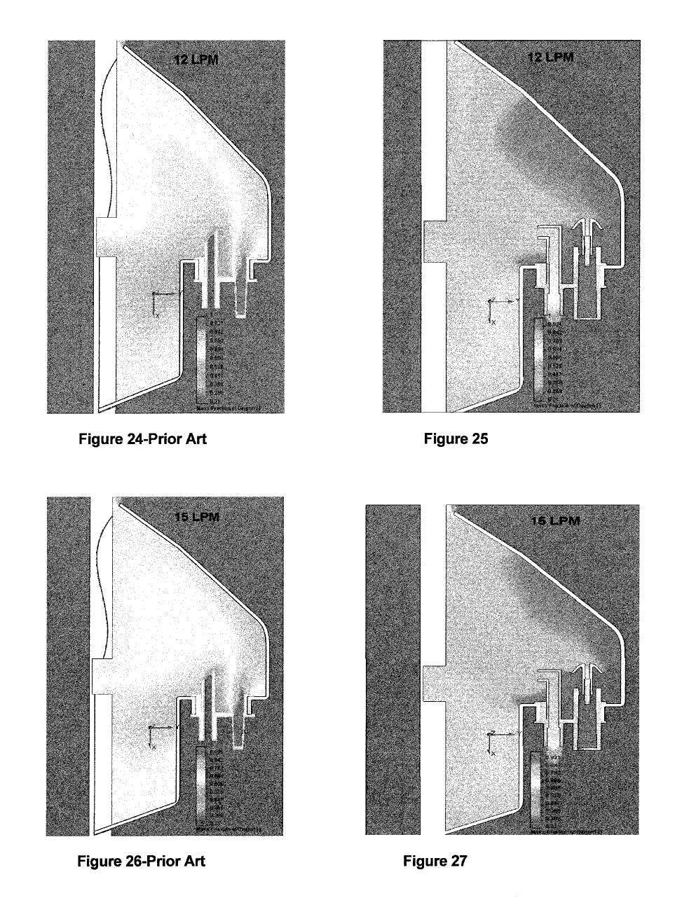

Flow simulations were generated by computer model, in which a prior art device was compared with an embodiment of the present invention. The prior art device comprised the Capnoxygen mask, manufactured by SouthMedic Inc. The results of this simulation are illustrated graphically in FIGS. 20-27, which illustrate simulated flow patterns within the mask interior at gas flow rates of between 4 and 15 LPM.

Various aspects of the present invention have been described above by reference to a detailed description of certain embodiments, features and experimental data thereof. It will be understood that these particulars are not intended to limit the scope of the invention, which includes departures, equivalents and other modifications to the particulars described above. The full scope of the invention will be understood from the present specification as whole, including the claims, and including equivalents of the elements described herein, as well as embodiments which may delete certain elements in a non-essential fashion.

1. A mask for delivery of a medical gas to a patient comprising a mask body having a rim for contacting the patient's face and an interior space configured to surround and enclose the patient's nose and mouth, and a gas diffuser structure projecting through said mask body into said interior space, said gas diffuser structure comprising a support, a gas tube within said support having an internal bore the axis of which is generally vertical when said mask body is upright, an inlet in said gas tube for communication with a source of pressurized gas, and an outlet from said gas tube within said interior space, and a baffle comprising a gas strike surface located over the outlet of said bore in the path of gas exiting said bore configured to diffuse and/or redirect said gas radially outwardly.

2. The mask of claim 1 wherein said baffle comprises a post mounted within said bore and a head supported by said post, an undersurface of said head comprising said gas strike surface.

3. The mask of claims 2 wherein said gas strike surface is concave.

4. The mask of claim 2 further comprising a support projecting radially inwardly within said bore to seat said post within said bore.

5. The mask of claim 4 wherein said support comprises a sleeve projecting upwardly from said bore to receive the post within said sleeve, said sleeve being inwardly spaced from the inside surface of said bore.

6. The mask of claim 4 wherein said baffle is removable from said bore.

7. The mask of claim 1 wherein said mask body comprises a forwardly protruding snout portion, said snout portion having a floor wherein said gas diffuser structure projects through said floor into the interior of said snout portion.

8. The mask of claim 1 further comprising a conduit for sampling exhaled breath from a user, said conduit including an inlet within the mask interior facing the user and an outlet for transmitting exhaled breath to an external gas monitor.

9. The mask of claim 8 wherein said conduit comprises a gas sampling tube projecting horizontally towards the user when the mask is upright, said gas sampling tube being positioned below said outlet and having an inlet radially displaced therefrom towards the user's face.

10. The mask of claim 9 wherein said inlet comprises a port within said mask body, said port communicating with said conduit.

11. The mask of claim 10 wherein said port is positioned below said outlet and radially displaced therefrom towards the user's face.

12. A kit of parts for a medical gas delivery mask, said kit comprising a mask body having a rim for contacting the patient's face and an interior space configured to surround and enclose the patient's nose and mouth, a gas diffuser structure for projecting through said mask body into said interior space, said gas diffuser structure comprising a support, a gas tube within said support having an internal bore the axis of which is generally vertical when said mask body is upright, an inlet in said gas tube for communication with a source of pressurized gas, and an outlet from said gas tube within said interior space, and a baffle comprising a gas strike surface located over the outlet of said bore in the path of gas exiting said bore configured to diffuse and/or redirect said gas radially outwardly.

13. The kit of claim 12 wherein said baffle comprises a post mounted within said bore and a head supported by said post, an undersurface of said head comprising said gas strike surface.

14. The kit of claim 13 wherein said gas strike surface is concave.

15. The kit of claim 13 further comprising a support projecting radially inwardly within said bore to seat said post within said bore.

16. The kit of claim 15 wherein said support comprises a sleeve projecting upwardly from said bore to receive the post within said sleeve, said sleeve being inwardly spaced from the inside surface of said bore.

17. The kit of claim 16 wherein said baffle is removable from said bore.

18. The kit of claim 12 wherein said mask body comprises a forwardly protruding snout portion, said snout portion having a floor wherein said gas diffuser structure projects through said floor into the interior of said snout portion.

19. The kit of claim 12 further comprising a conduit for receiving exhaled breath from a user, said conduit including an inlet within the mask interior facing the user and an outlet for transmitting exhaled breath to an external gas monitor.

20. The kit of claim 19 wherein said conduit comprises a tube projecting horizontally towards the user when the mask is upright, said tube being positioned below said outlet and having an inlet radially displaced therefrom towards the user's face.

21. The kit of claim 20 wherein said inlet comprises a port within said mask body, said port communicating with said conduit.

22. The kit of claim 21 wherein said port is positioned below said outlet and radially displaced therefrom towards the user's face.

23. The kit of claim 12 comprising a plurality of baffles having different configurations.

24. A method for delivering gas to a patient, comprising providing a mask which defines a substantially enclosed space over the patient's nose and mouth region including a rim contacting the patient's face around the nose and mouth region, delivering a pressurized gas into the mask interior in a generally upright direction when the mask is upright and deflecting the upwardly rising gas flow radially outwardly in a horizontal plane.

25. The method of claim 24 wherein said gas flow is deflected by introducing the gas flow through a vertical conduit into the mask interior, and positioning a baffle over the outlet of the conduit within the mask interior, whereby the diffuser has a gas strike surface facing the gas outlet.
